(19) United States
(12) Patent Application Publication
(10) Pub. No.: US 2011/0236864 A1
(43) Pub. Date: Sep. 29, 2011

(54) Memory test for alzheimer's disease

(76) Inventor: John Wesson Ashford, Redwood City, CA (US)

(21) Appl. No.: 12/931,919

(22) Filed: Feb. 14, 2011

Related U.S. Application Data
(60) Provisional application No. 61/339,663, filed on Mar. 5, 2010.

(51) Int. Cl. A61B 5/00

(52) U.S. Cl. 434/236

(57) ABSTRACT

a method for assessing memory in a subject include the steps of presenting to the subject a list of items to be retrieved from memory by the subject, having the subject recognize the presented items from memory, determining the subject's response speed to each of the recognized repeated items and analyzing a plurality of the response speeds for the recognized repeated items. The items which presented to the subject are intermixed with repetitions of the items being tested for recognition. The subject is tested to determine if he recognizes each repeated item as being a repeated item. The response speed for each of the recognized repeated item is the time required between when the subject is shown a repeated item and when the subject responds that he recognizes the repeated item.

The application is a continuation-in-part of the provisional application filed under Ser. No. 61/339,663 on Mar. 1, 2010.

### BACKGROUND OF THE INVENTION

1. Field of the Invention

This invention relates to a method for increasing the usefulness, sensitivity and specificity of tests that measure memory and facets of memory, including learning, retention, recall and/or recognition. Specifically, the sensitivity and specificity of such tests are enhanced by selectively weighting the value of specific items recalled by the test subject, either by weighting such items within any specific testing trial or across numerous testing trials. Also disclosed are various methods of reducing ceiling effects in memory tests. The invention also provides improved tests which employ item-specific weighting for the diagnosis of Alzheimer's Disease and other dementia characterized by memory impairment, as well as a method of screening for and evaluating the efficacy of potential therapeutics directed to the treatment of such dementia.

2. Description of the Prior Art

U.S. Pat. No. 7,314,444 teaches a method which assesses either episodic memory or semantic memory in a subject. The method screens for an agent directed to treating, slowing down the progress of, attenuating the symptoms of, or preventing dementia characterized by episodic memory impairment. Additionally, the method screens for an agent directed to treating, slowing down the progress of, attenuating the symptoms of, or preventing dementia characterized by semantic memory impairment. Finally, the method measures semantic memory in a subject.

It is estimated that, over the next twenty years, one in every five persons will be over the age of 65. With this new demographic profile will come an increase in a wide variety of age-related conditions, such as dementia, including Alzheimer's disease. Dementia is a syndrome of progressive decline, in multiple domains of cognitive function that eventually leads to an inability to maintain normal social and/or occupational performance. At present, AD is the most common type of dementia, afflicting approximately 4 million Americans. One in ten persons over the age of 65, and nearly half of those over the age of 85, suffer from AD, and AD is the fourth leading cause of death in the U.S. The cost to American society is estimated to be at least $100 billion every year, making AD the third most costly disorder of aging.

Early identification is critical in progressive conditions such as AD, because earlier treatment may be more effective than later treatment in preserving cognitive function. Furthermore, early detection may allow time to explore options for treatment and care. Nevertheless, early detection is compromised by the failure of many patients to report to their treating physicians such early symptoms of AD as memory lapses and mild, but progressive, deterioration of specific cognitive functions, e.g., language (aphasia), motor skills (apraxia), and perception (agnosia). In addition, studies have documented the difficulty experienced by even well-trained health care professionals in correctly diagnosing AD and other forms of dementia (1). Accordingly, a simple, sensitive, reliable, and easily-administered AD diagnostic test would be of great assistance in targeting individuals for early intervention.

The earliest manifestation of AD is often memory impairment, which is a requirement in each of the two sets of criteria for diagnosis of dementia that are commonly used—the National Institute of Neurological and Communicative Disorders and Stroke/Alzheimer's Disease and Related Disorders Association (NINCDS/ADRDA) criteria, which are specific for Alzheimer's disease, and the American Psychiatric Association's Diagnostic and Statistical Manual of Mental Disorders, Fourth Edition (DSM-IV) criteria, which are applicable for all forms of dementia. Therefore any test for AD or dementia associated with memory impairment should be most sensitive for the early detection of memory impairment. Conventional memory tests are not optimal for the detection of mild dementia or the early stages of Alzheimer's disease. Some of these tests are inappropriately sensitive to the patient's educational level (White and Davis, Journal of General Internal Medicine, Volume 5, pages 438-445, 1990: McDowell and Kristjansson, Mental Status Testing, in Measuring Health: a guide to rating scales and questionnaires, 1996:287 334). They may also fail to test for certain types of memory loss that are typical of early dementia or Alzheimer's Disease, as well as fail to reflect whether compounds or therapy administered to treat dementia are having the desired effect. These tests also often suffer from a high rate of false negatives (low sensitivity) or false positives (low specificity).

Although there are many variations, a typical memory test is structured as follows. First, the tester presents to the subject a number of items (i.e., bowl, zebra, orange and anger) to be recalled from memory. The items may be presented orally, in writing, in pictures, or by any other suitable means. Sometimes the subject is also supplied with a cue associated with each or some of the items on the list. The cue typically is the category that encompasses the associated item or defines an aspect of the associated item, i.e., the cue “animal” or “stripes” might be presented in association with the item “zebra”. Certain conventional paired associate tests use unrelated nouns as cues for test items.

The presentation of items in association with a cue, where the subject must first identify the item from the cue, is known as “controlled learning”. Controlled learning is used in memory tests to assure the attention of the subject and the equal processing of all the items in a list. In addition, it shows that the subjects can identify items from their cues, and induces encoding specificity, by providing cues at the time of encoding information that can later be used to aid recall. The identification of items by matching the items with the associated cues verifies that the required processing was performed by the subject.

In the next step, the subject is asked to recall the items presented to him in the list, either from memory without presentation of the associated cue, known as “free recall”, or after being presented with the associated cue, known as “cued recall.” Cued recall may be used selectively to facilitate recall of those items not first recalled by free recall (without any cues).

Conventional memory tests are not optimal for the detection of mild dementia or the early stages of Alzheimer's disease. Some of these tests are inappropriately sensitive to the patient's educational level (2). They also may fail to test for certain types of memory loss that are typical of early dementia or AD. In addition, they may fail to reflect whether compounds or therapies that are administered to treat dementia have the desired effects. Furthermore, these tests frequently suffer from a high rate of false positives (low specificity).

Essentially all tests of memory, including tests of learning, retention, recall and/or recognition (hereinafter referred to as “memory tests”), involve serial-processing memory. “Serial-processing memory” acknowledges the limitations in a subject's input and output processing, which limitations permit the subject to process only one item at a time during input (receiving information) and output (recalling or retrieving information). In this sense, “memory” comprises units of received information and recalled information. In most tests of serial-processing memory, multiple items are presented serially to a subject, one item at a time, until all of the items have been presented (“serial presentation”), and the subject reports the remembered items serially, one item after another, in the order in which they were originally presented (“serial recall”). Items may be presented verbally, visually, or by any other suitable means.

Although there are many variations, a typical memory test consists of two main steps. First, the tester presents to the subject a number of items (e.g., bowl, zebra, orange, anger) to be recalled from memory. The items may be presented orally, in writing, in pictures, or by any other suitable means. Sometimes, the subject is also supplied with a cue associated with each or some of the items on the list. Typically, the cue is the category that encompasses the associated item, or that defines an aspect of the associated item. For example, the cue “animal” or “stripes” might be presented in association with the item “zebra”. Certain conventional, paired-associates tests use unrelated nouns as cues for test items.

The presentation of items in association with cues, wherein the subject first must identify an item from its cue, is known as “controlled learning”. Controlled learning is used in memory tests to ensure the attention of the subject and the equal processing of all of the items in a list. In addition, controlled learning demonstrates that the subject can identify items from their cues. Controlled learning also induces encoding specificity, by providing cues at the time of encoding information that later can be used to aid recall. The correct identification of items, by matching the items with their associated cues, verifies that the required processing was performed by the subject. Controlled learning may be performed by normal individuals and by those with dementia or others type of cognitive decline.

In the next step, the subject is asked to recall the items presented to her in the list, either from memory, without presentation of the associated cue, in any order (free recall), or following presentation of the associated cue (cued recall). Cued recall may be used selectively to facilitate recall of those items not first recalled by free recall (recall in any order, without presentation of any cues).

One variation of the standard memory test involves an additional step for “controlled rehearsal”, wherein the subject is instructed to repeat each item as it is presented. Alternately, the subject may be asked to repeat the preceding item as the current item is presented, or the subject may be instructed to repeat both the preceding item and the current item. As with controlled learning, controlled rehearsal ensures the attention of the subject and the equal processing of all items, and demonstrates that the required processing was performed by the subject. Some memory tests, particularly tests of delayed recall or forgetfulness, utilize interference delays between memory trials. Interference delays are periods of time between memory trials, wherein an unrelated task (e.g., counting, spelling) is performed by the subject to prevent rehearsal. As another alternative, the tester may maintain a constant number of items to be learned by the subject. This step, referred to as “contingent learning”, can be accomplished by adding new items to the list as the old items are learned. “Controlled reminding” refers to an additional step wherein the subject is reminded of items that were not recalled during each trial. In free-recall memory tests, the subject is not reminded of the items that were not recalled until after he has been given the opportunity to recall as many items as possible. In the case of cued recall, the subject is reminded of the item before the next cue is presented. Controlled reminding may be either “selective reminding”, wherein the subject is reminded each time the item is not recalled, or “restricted reminding”, wherein the subject is reminded only until the item is recalled once, either with or without presentation of the item.

Memory tests known in the art include various combinations of the foregoing elements and steps. For example, the memory component of the Free and Cued Selective Recall Test includes tan initial controlled-learning step, wherein the patient first must identify items from their associated cues. The patient then must recall as many of the test items as possible (typically sixteen) from their associated category cues. Following a brief interference delay, the patient is then asked to recall as many of the presented items as she can by free recall (i.e., recall in any order, without the associated cues), followed by cued recall for items not remembered by free recall. If there are multiple trials, the subject is then selectively reminded of missed items (i.e., reminded each time an item is not recalled) before the next recall trial. The score for total recall is the total of uncued responses and cued responses, with each response (whether cued or uncued) worth one point.

Conventional memory tests are scored by tallying the total number of items recalled from a list of items previously presented to the subject, either within any one testing trial or across many testing trials. Each item recalled is accorded the same weight—a method known as “unit counting” or “unweighted counting”—so that a subject recalling items 1 to 5 of a ten-item list would be judged to have the same measure of memory as a subject who recalled items 6 to 10 of the same ten item list. Furthermore, a subject recalling items 1 to 5 of a ten-item list in a first trial, and items 6 to 10 of the list in a second trial, would be considered to have the same measure of memory as a subject who recalled items 1 to 5 in the first and second trials, but could not recall items 6 to 10 at all. Memory tests utilizing this type of unweighted counting assume that all of the items presented and retrieved are equal in value, i.e., that the probability of encoding, learning, and retaining any single item is equal to the probability of encoding, learning, and retaining any other items. However, items in a presented list differ in likelihood of recall, depending upon a number of factors. Serial processing at input and output often results in “serial-position effects”, which are differences in the frequency of recall among list items due to the order in which the items are presented. In this regard, some items are “easier” or “more difficult” to recall, depending upon the order in which such items are presented to the subject. These serial-position effects are illustrated for test groups in serial-position curves, which are graphs that show the percentage of subjects recalling the items of a list versus the order in which the items are presented or recalled.

Serial-position effects demonstrate that the probability of recall may be affected by the order in which test items are presented (“presentation order”), or the order in which test items are recalled (“recall order”), or both. Important serial-position effects include “primacy”, in which there is a higher recall of earlier presented items, and “recency”, in which there is a higher recall of the items most recently presented. An analysis of serial-position effects is important because the manifestation of certain serial-position effects (or the lack thereof) may be associated with dementia. For instance, recall by normal aged subjects is characterized by both primacy and recency effects, whereas recall by aged subjects with AD is characterized only by recent effects.

Additionally, unweighted counting disregards qualitative differences in memory impairment, e.g., where impairment in total memory is a result of deficiencies in a particular stage or facet of the memory process, such as a deficiency in encoding information, learning information, or retaining information. Such qualitative differences may be essential for the diagnosis of AD or dementia characterized by memory impairment, and may be instrumental in ensuring the appropriate targeting of, and evaluation of the efficacy of, therapeutics directed to the treatment of AD or early dementia.

In view of the foregoing, the measurement of memory by unweighted counting (unit weighting) may not be justified. Although unweighted counting provides a lower bound for memory performance, it sacrifices statistical power by disregarding essential information about the serial-position characteristics of retrieved items (including probability of retrieval), and about the various processes involved in memory (including encoding, learning, and retention). A method of measuring memory that preserves information about the serial-position effects of retrieved items, or that recognizes deficiencies in certain elements of total memory, would improve the assessment of memory performance, aid in the earlier diagnosis of AD and dementia, and permit the sophisticated screening of therapeutics directed to the treatment of AD and dementia.

U.S. Pat. No. 6,964,638 teaches a method which measures the cognitive performance of an individual. The individual completes at least one cognitive test with at least one testing protocol. The result of at least one cognitive test is stored in a computer readable media. A reliable change technique is applied to calculate a reliable change measure. The reliable change measure is a statistically meaningful inference of a neurological pathology. The reliable change technique uses at least one baseline of the individual. The neurological pathology is a dementia which is global-diffuse cerebrum disorder. The dementia is selected from the group consisting of either Alzheimer's pre-senile dementia or Alzheimer's senile onset dementia.

Cognitive function can be impaired by physical insults such as radiation or chemical exposure. Measuring changes in cognitive function is thus an important way to quantify the adverse cognitive effects of such insults. Intentional exposure to electro-magnetic radiation (including electricity) may cause cognitive impairment. For example, electro-convulsive therapy, used in psychiatric treatments, is acknowledged in the art to be beneficial to certain patients. It is, however, difficult to quantify how beneficial it is, as the cognitive impairment effects of ECT are difficult to measure. Similarly, radiation therapy (as, for example, for a cancer patient) is acknowledged to create memory loss. It is, however, difficult to measure this memory loss. Other electromagnetic exposure, such as exposure to laser light, or even excess exposure to sunlight, may impair cognition. Similarly, accidental exposure to radiation may impair cognition. For example, it has been argued that cellular telephones, by exposing the user's brain to electromagnetic radiation, can impair cognition. The accuracy of this fear is, however, difficult to measure. Other types of physical intervention or therapy may affect cognition. For example, vaccines, gene therapy and other kinds of biological implants (e.g., transplants, stem cell implants and fetal cell implants), exposure to toxins, molds or chemicals in the environment (including, for example, Gulf War syndrome and other exposure to pollution or weapons chemical, nuclear, conventional or biological), ingestion of vitamins, herbs, over the counter compositions or homeopathic preparations or other ingested things, may impair cognitive function, but measuring such effects until now has been prohibitively difficult. There has thus been a need for a way to measure cognitive function more precisely, to quantify changes in cognitive function. There is a point to be made about serial presentation of information, but the point of Memtrax is that presentation and assessment do not have to be discrete epochs. It is important to discuss and address change, such as either Gulf War Syndrome (or Gulf War Illness) or another event is traumatic brain injury. The important issue is that the assessment of normal memory with respect to aging and the determination when a significant loss of function has occurred or a significant change has occurred as an indicator of or prelude to the development of a pathological state.

U.S. Pat. No. 5,913,310 teaches a method which diagnoses and treats psychological and emotional disorders using a microprocessor based video game. Examples include “schizophrenia, depression, hyperactivity, phobias, panic attacks, anxiety, overeating, and other psychological disorders” such as “personality disorders, obsessive-compulsive disorders, hysteria, and paranoia.”

U.S. Pat. No. 5,872,713 teaches an analyte testing system with test strips. These test strips are effective to assay for certain chemical changes in a patient, but have not been used to assay cognitive dysfunction or impairment.

U.S. Pat. No. 5,827,179 teaches a personal computer card for collecting biological data. A portable computer card is used with either an air pressure transducer or a “biological data receiver.” The biological data receiver “can be adapted to receive biological data from a pulse oximetry sensor” or “from an ECG sensor.” This system may be effective to assay for certain physical changes in a patient such as pulmonary function, but it has not been used to assay cognitive dysfunction or impairment.

U.S. Pat. No. 5,715,451 teaches a system which constructs formulae for processing medical data. Rather than providing a prepared statistical analysis package a computer interface constructs statistical and other mathematical formulae to ease the analysis of clinical data.

U.S. Pat. No. 5,778,882 teaches a health monitoring system that “tracks the state of health of a patient and compiles a chronological health history using a multi-parametric monitor which automatically measures and records a plurality of physiological data from sensors in contact with the patient's body,” wherein “[t]he data collected is not specifically related to a particular medical condition” such as cognitive dysfunction.

Not disclosed in the above-cited prior art is a non-invasive, computerized system to accurately measure cognitive function and changes in cognitive function over time. There is a need for such an invention that is easy to administer, rapid, less expensive, and accurate enough to enable assessment of the cognitive impairment impact of a wide variety of agents or environments.

U.S. Pat. No. 6,306,086 teaches memory tests using item-specific weighted memory measurements and uses thereof which increase the usefulness, sensitivity and specificity of tests that measure memory and facets of memory, including learning, retention, recall and/or recognition. Ashford first described these issues for studying items and applying item analysis to Alzheimer's disease and dementia in 1989. See Ashford J W, Kolm P, Colliver J A, Bekian C, Hsu L N. Alzheimer patient evaluation and the mini-mental state: item characteristic curve analysis, Journal of Gerontology 1989 September, Volume 44(5), pages 139-46. See also http://www.medafile.com/jwa/JWA1989.pdf.

Another useful citation for the need to screen for Alzheimer's disease is Ashford J W, Borson S, O'Hara R, Dash P, Frank L, Robert P, Shankle W R, Tierney M C, Brodaty H, Schmitt F A, Kraemer H C, Buschke H. Fillit H, “Should older adults be screened for dementia? It is important to screen for evidence of dementia,” Alzheimer's & Dementia (2007) April, volume 3, pages 75-80. See also http://www.medafile.com/jwa/JWAA&D07.pdf.

A good review of tests to screen to memory problems, dementia and Alzheimer's disease is Ashford J W, “Screening for Memory Disorder, Dementia, and Alzheimer's disease,” Aging Health (2008), Volume 4(4), pages 399-432. See also http://www.medafile.com/jwa/Ashford AH08.pdf.

There is a paper in review by the Journal of Gerontology that reviews a lot of material advocating the MemTrax type of memory testing for AD.

Specifically, the sensitivity and specificity of such tests are enhanced by selectively weighting the value of specific items recalled by the test subject, either by weighting such items within any specific testing trial or across numerous testing trials. Also disclosed are various methods of reducing ceiling effects in memory tests. The invention also provides improved tests which employ item-specific weighting for the diagnosis of Alzheimer's Disease and other dementia characterized by memory impairment, as well as a method of screening for and evaluating the efficacy of potential therapeutics directed to the treatment of such dementia. This is a method for increasing the usefulness, sensitivity and specificity of tests that measure memory and facets of memory, including learning, retention, recall and/or recognition. Specifically, the sensitivity and specificity of such tests are enhanced by selectively weighting the value of specific items recalled by the test subject, either by weighting such items within any specific testing trial or across numerous testing trials. There are various methods of reducing ceiling effects in memory tests and improved tests which employ item-specific weighting for the diagnosis of Alzheimer's Disease and other dementia characterized by memory impairment, as well as a method of screening for and evaluating the efficacy of potential therapeutics directed to the treatment of such dementia.

It is estimated that over the next 20 years, one in every five persons will be over the age of 65. With this new demographic profile will come an increase in a wide variety of age-related conditions, including Alzheimer's disease (“AD”) and other forms of dementia. Dementia is a syndrome of progressive decline in multiple domains of cognitive function, eventually leading to an inability to maintain normal social and/or occupational performance. At present, AD is the most common form of dementia, afflicting approximately 4 million Americans. One in ten persons over the age of 65 and nearly half of those over the age of 85 suffer from AD, and AD is the fourth leading cause of death in the U.S. The cost to U.S. society is estimated to be at least $100 billion every year, making AD the third most costly disorder of aging.

A portable electronic cognometer includes a memory monitor and a concentration monitor. Repeated testing of memory and concentration is needed to identify declining cognitive function that may require medical evaluation for dementia, delirium, other medical or psychiatric illness, or the cognitive side-effects of medications. Repeated cognitive monitoring is not commonly carried out even in medical settings, let alone at home, due to lack of a device for automated, easily repeated testing.

Easily repeated testing also makes it possible to determine any individual's best (and worst) performance at baseline, so that the individual's memory and concentration in the future can be evaluated by comparison with his or her own previous performance, rather than only by reference to less sensitive general norms obtained from the performance of other more or less similar persons.

Automated testing should provide reliable, rapid, and automatic administration, scoring, and reporting, so that repeated testing can be carried out reliably in precisely the same way as frequently as desired, at home as well as in medical settings. This permits self-testing by the general public, and monitoring of patients with Alzheimer's disease or other cognitive impairment, at home as well as in medical offices, clinics, emergency rooms, hospital wards, psychiatric facilities, or nursing homes.

Tests of memory and concentration should be designed to elicit maximum performance on rigorous but brief and easily repeated tests of sensitive, early, and prominent indicators of cognitive impairment. These tests will be regarded as ecologically valid and appropriate only if they check functions that everyone is expected to be able to perform in ordinary life; for example, everyone is expected to be able to remember a telephone number or copy a sequence of digits. The tests of memory and concentration must require only the simplest of responses, e.g., pressing numbered keys, so that appropriate responses can be obtained from all but the most severely impaired persons. Preferably these tests should be self-paced to compensate for the cognitive slowing often present in aged or cognitively impaired persons. To identify excessive slowing that may be an early indication of impaired cognitive processing, response speed may be measured and reported.

It is essential that such tests measure only the ability in question and not be affected by other considerations. For example, if a person has vision or reading problems which prevent him from seeing or understanding what he does see, his subsequent inability to reproduce a number displayed does not reflect on his memory. Similarly, if he has manual dexterity problems which interfere with his reproducing a displayed number on a keyboard, his failure to key in a number which he was supposed to memorize does not reflect on his memory. Thus it is critical that any cognitive test isolate the cognitive ability being tested and, even if it does not test that ability alone—for other factors always come into play—the test should at least evidence the other factors at play.

A reliable, rapid and automatic administration, scoring and reporting test for self-testing at home or elsewhere is desirable as is a device which provides for ecologically valid and appropriate testing and which effectively isolates for testing the cognitive ability to be tested, even in aged or infirm users.

U.S. Pat. No. 5,230,629 teaches a cognitive speedometer for the assessment of cognitive processing speed which includes a display screen, a keyboard, and a processor for generating original data and displaying on the screen the original data for copying by a user on the keyboard. Only if the user copies the displayed original data correctly, the processor generates and displays on the screen different data on which the user is to perform a unit cognitive operation and then enter the resultant data on the keyboard, the resultant data having the same characters as the original data. Only if the user enters the correct resultant data, the processor determines the time required for the user to perform the unit cognitive operation.

U.S. Pat. No. 4,770,636 teaches a cognometer useful in the repeated testing of memory and concentration, as needed to identify declining cognitive function that may require medical evaluation for dementia, delirium, other medical or psychiatric illness, or the cognitive side-effects of medications. The apparatus therein described permits repeated cognitive monitoring to be carried out not only in a medical setting, but also alone at home, through the provision of a device for automated, easily repeated testing. The cognitive functions determined by the apparatus set forth therein are memory and concentration, rather than the speed of the cognitive functions. While memory and concentration are particularly useful foci in many instances, particularly those involving the elderly or the severely affected, in other instances the primary focus should be on the speed with which a cognitive function is performed. For example, airplane pilots, racing car drivers and many others are required to make decisions not only accurately, but also rapidly.

The cognometer provides self-based testing expressly to compensate for the cognitive slowing often present in aged or cognitively impaired persons, although response speed was measured and reported in order to enable identification of excessive slowing which could be an early indication of an impaired cognitive processing. By way of contrast, the present invention is directed to a cognitive speedometer for the assessment of cognitive processing speed with fineness and sensitivity. This is accomplished by determining the cognitive processing speed independently of the time required to physically respond to the test stimulus. Such a test must measure only the ability in question and not be affected by other considerations, such as the physical functions of the individual being tested. For example, if a person has manual dexterity problems which interfere with his reproducing a displayed number on a keyboard, an excessive time to key in an answer in response to a presented arithmetic problem does not necessarily reflect on the speed of his cognitive processing. Thus, it is critical that any cognitive speedometer test isolate the cognitive function.

Automated testing should provide reliable, rapid, and automatic administration, scoring, and reporting so that repeated testing can be carried out reliably in precisely the same way as frequently as desired, at home as well as in medical settings. This permits self-testing by the general public, and monitoring of patients with suspected cognitive impairment, at home as well as in medical offices, clinics, emergency rooms, hospital wards, psychiatric facilities, or nursing homes. In order for the test to have validity as an indication of cognitive processing speed, as independent as possible of intelligence, education and the like, the cognitive function to be performed during the test should be short, simple and capable of being performed in only one manner. To this end, the apparatus should test a unit or single cognitive operation as opposed to a complex set of cognitive operations which might be strongly affected by the intelligence of the test taker, the manner in which he approached the problem or performed the arithmetic operation, etc. For example, certain arithmetic problems can be solved more easily and faster by successive approximation until the right answer is obtained than through the set of arithmetic operations intended by the problem framer—e.g., a long division problem or a solution to a complex equation.

The measurement of cognitive speed by conventional techniques—such as comparison of simple and multiple choice reaction times, Sternberg's memory scanning paradigm, or speed of mental rotation—have not proven to be entirely satisfactory. Some of these methods appear to measure the time needed to carry out more than one cognitive operation because they require a cognitive decision in addition to the operation(s) involved in their mental comparisons. Some of these rely on yes/no responses which allow guessing with a high probability of correct guessing rather than accurately measuring cognitive speed. Thus, the need remains for a cognitive speedometer which is designed to measure the speed of a single cognitive operation without reflecting the time needed to enter the result, requires specific numerical responses that cannot be guessed or anticipated, and does not require the kind of decisions needed for yes/no responses. The measures provided by such a cognitive speedometer should reflect the speed of a single cognitive operation, without additional variance due to decision latencies, and without contamination by rapid guesses, so that the latencies should provide more accurate statistics, and the fastest responses should provide a more accurate measure of maximum cognitive speed.

U.S. Pat. No. 7,070,563 teaches a method which increases the usefulness, sensitivity and specificity of tests that measure memory and facets of memory, including learning, retention, recall and/or recognition.

Specifically, the sensitivity and specificity of such tests are enhanced by selectively weighting the value of specific items recalled by the test subject, either by weighting such items within any specific testing trial or across numerous testing trials. Also disclosed are various methods of reducing ceiling effects in memory tests. The invention also provides improved tests which employ item-specific weighting for the diagnosis of Alzheimer's Disease and other dementia characterized by memory impairment, as well as a method of screening for and evaluating the efficacy of potential therapeutics directed to the treatment of such dementia.

Variations of memory tests include “controlled rehearsal”, which refers to a step wherein the subject is instructed to repeat each item as it is presented. Alternately, the subject may be asked to repeat the preceding item as the current item is presented, or the subject may be instructed to repeat both the preceding item and the current item. As with controlled learning, controlled rehearsal assures attention and equal processing of all items, and shows that the required processing was performed.

Some memory tests, particularly tests of delayed recall or forgetfulness, utilize interference delays between memory trials. Interference delays are periods of time between memory trials wherein an unrelated task is performed by the subject to prevent rehearsal by the subject. Common tasks performed to prevent rehearsal include having the subject count, spell, or perform a simple unrelated task.

“Controlled reminding” refers to a step wherein the subject is reminded of items that were not recalled during each trial. In free recall memory tests, the reminding would not occur until after the subject is given the opportunity to recall as many items as possible. In the case of cued recall, the subject is reminded of the item before the next cue is presented. Controlled reminding may be either “selective reminding”, wherein the subject is reminded each time the item is not recalled, or “restricted reminding”, wherein the subject is reminded only until the item is recalled once, either with or without presentation of the item. “Contingent learning” refers to maintaining a constant number of items to be learned. This can be done by adding new items as the old items are learned.

Memory tests known in the art include various combinations of the foregoing elements. For example, the memory component of the Free and Cued Selective Recall Test (“FCRST”) is comprised of an initial controlled learning step, where the patient must first identify items from their associated cues. The patient must then recall sixteen tests items from their associated category cues. Following a brief interference delay, the patient is then asked to recall as many of the presented items as she can by free recall, i.e., without the associated cues, followed by cued recall for items not remembered by free recall. If there are multiple trials, then the subject is selectively reminded (i.e., reminded each time an item is not recalled) of missed items before the next recall trial. The score is the total of uncued responses and cued responses, with each response (whether cued or uncued) worth one point.

Conventional memory tests are scored by tallying the total number of items recalled from a list of items previously presented to the subject, either within any one testing trial or across many testing trials. Each item recalled is accorded the same weight (“unit counting” or “unweighted counting”), so that a subject recalling items 1 to 5 of a ten item list would be judged to have the same measure of memory as a subject who recalled items 6 to 10 of the same ten item list. Further, a subject recalling items 1 to 5 of a ten item list in a first trial and items 6 to 10 in a second trial would be considered to have the same measure of memory as a subject who recalled items 1 to 5 in the first and second trials, but could not recall items 6 to 10 at all. Memory tests utilizing this type of unweighted counting assume that all the items presented and retrieved are equal in value, i.e., that the probability of encoding, learning, and retaining any single item is equal to the probability of encoding, learning and retaining any other items.

However, items in such a list differ in likelihood of recall, depending on a number of factors. Serial processing at input and output almost always result in “serial position effects”, which are differences in the frequency of recall among list items due to the order in which the items are presented. Simply put, some items are “harder” or “easier” to recall depending upon the order such items are presented to the subject. These serial position effects are illustrated for groups in serial position curves, which are graphs that show the percentage of subjects recalling the items of a list versus the order in which the items are presented or recalled. These serial position effects show that the probability of recall is affected by the order in which the items are presented (“presentation order”), or the order in which the items are recalled (“recall order”), or both. Important serial position effects include primacy (higher recall of earlier presented items) and recency (higher recall of the most recently presented items). Analysis of serial position effects is important because the display of certain serial position effects (or the lack thereof) may be associated with dementia. For instance, recall by normal aged subjects is characterized by primacy effects as well as recency effects, but recall by aged subjects with AD is characterized only by recency effects.

Also, unweighted counting ignores qualitative differences in memory impairment, that is, whether impairment in total memory is a result of deficiencies in a particular stage or facet of the memory process, namely a deficiency in encoding information, learning information or retaining information. Such qualitative differences may be essential for the diagnosis of AD or dementia characterized by memory impairment and to appropriately target and evaluate the efficacy of therapeutics directed to the treatment of AD or early dementia.

As a result, measuring memory by unweighted counting (i.e., unit weighting) may not be justified. Although unweighted or unit counting provides a lower bound for memory performance, it sacrifices statistical power by ignoring essential information about the serial position characteristics (probability of retrieval) of retrieved items and about the various processes involved in memory (i.e., encoding, learning and retention). A method of measuring memory that preserves information about the serial position effects of retrieved items or that pinpoints deficiencies in certain elements of total memory would improve the assessment of memory performance, aid in the earlier diagnosis of dementia and AD, and permit sophisticated screening of therapeutics directed to the treatment of AD or dementia.

The inventor hereby incorporates all of the above referenced patents into his specification.

### SUMMARY OF THE INVENTION

The present invention is a method for assessing memory in a subject that includes the steps of presenting to the subject a list of items to be retrieved from memory by the subject, having the subject recognize the presented items from memory, determining the subject's response speed to each of the recognized repeated items and analyzing a plurality of response speeds for the recognized repeated items.

In a first aspect of the present invention the items presented to the subject are intermixed with repetitions of said items being tested for recognition

In a second aspect of the present invention the subject is tested to determine if he recognizes each repeated item as being a repeated item.

In a third aspect of the present invention the response speed for each of the recognized repeated item is the time required between when the subject is shown a repeated item and when the subject responds that he recognizes the repeated item.

Other aspects and many of the attendant advantages will be more readily appreciated as the same becomes better understood by reference to the following detailed description and considered in connection with the accompanying drawing in which like reference symbols designate like parts throughout the figures.

The features of the present invention which are believed to be novel are set forth with particularity in the appended claims.

### DESCRIPTION OF THE DRAWINGS

FIG. 1 is a photograph of a slide show for MEMTEST according to the present invention.

FIG. 2 is a table showing the results of MEMTEST of FIG. 1.

FIG. 3 shows the relationship between age and mean hit rate and mean correct rejection rate in 868 individuals on the VNBT with each data point representing the performance of an individual participant.

FIG. 4 shows the relationship between discriminability (d′) and age on the VNBT with each data point representing the score (d′) of an individual participant. One individual whose score was unusually poor (d′=−2.64) was removed from the plot. The dashed line shows an inverted exponential regression curve which was initially fitted to 4 minus the d′ values.

FIG. 5 shows the relationship between discriminability performance (d′) and age in 868 individuals on the VNBT with numbers inside the bars indicating the group n. The bars show the mean discriminability score for each age group and brackets show SEM.

FIG. 6 shows the same data is shown with brackets showing one standard deviation on either side of the mean.

FIG. 7 shows the relationship between discriminability performance (d′) and education in 868 individuals on the VNBT with numbers inside the bars indicating the group n. The bars show the mean discriminability score for each age group and brackets show SEM.

FIG. 8 shows the relationship between the number of intervening items (between initial and first repeat presentations) and percent correct on those items across 868 individuals on the VNBT with the brackets showing SEM. Each item was shown for 5 seconds so the correspondence with the temporal interval can be calculated.

FIG. 9 shows the relationship between percent correct and item repetition in 868 individuals on the VNBT when eleven items were shown three times during the test. Recognition performance increased between the second and third presentation (average number of intervening items=21.1, range; 10-36 items). A paired t-test demonstrated that the difference was significant (p<0.005).

### DESCRIPTION OF THE PREFERRED EMBODIMENT

Memtrax Test consists of specifications for tests and games (the TEST) designed to measure cognition. The specific aspect of cognition assessed by the TEST is retentive memory, the mental function that most specifically deteriorates with increasing age and that declines in the time before and during a diagnosis of dementia. The TEST also assesses related aspects of cognition. The TEST is a brief slide show, administered by a computer (in any format, computer-program, or platform) which displays a series of visual image stimuli (pictures or words) and records the performance of tested subjects on each image.

A major aspect of cognition is memory. Memory involves the retention of perceived information, whether it is for an immediate circumstance (attentive memory), preserved for an indefinite period of time after the immediate circumstance has changed (retentive memory), or maintained for long periods of time, days, weeks, months, after the information was perceived (persistent memory). The earliest sign of Alzheimer's disease is generally considered an impairment of retentive memory, and this type of memory is also known to deteriorate substantially with the normal aging process.

1) Retentive Memory—Information Retention after distraction (minutes to hours, short-term memory, declarative memory).

2) General Attention—Attention to the task and the information presented.

3) Perceptual Attention—Attention to information details, which may include discriminating differences between pictures or words.

4) Recognition Reaction Time—Time to perceive an item of information and react according to the instructions of the test.

5) Attentive Memory—Information retention before distraction (for the duration of attention without distraction, e.g., before an intervening stimulus, immediate memory, working memory).

6) Persistent Memory—Information that is maintained for long periods of time (days to years, long-term memory, semantic memory)

The TEST displays a series of images and a portion of the images are repeated. The novel aspect of the TEST is that duplicated images are interspersed with images being shown for the first time. As the series advances, the individual being tested must make a binary decision when each image appears, whether the image is a target (e.g., a repeated image that is recognized) or a non-target (e.g., a new image to be observed and retained).

Memory performance is reflected in the percent of previously shown pictures that are recognized. Validity and attention are indicated by the percent of pictures shown for the first time which are perceived as being new images. Recognition reaction time can be measured. Images are selected to test specific aspects of perception and memory and may be pictures or words. The attributes of the images (e.g., nameable, memorable) can be varied to assess a broad range of cognitive abilities. The difficulty of the TEST may be adjusted to test retentive memory across a broad range of ability and with narrow precision. Variations of the TEST can more specifically assess general attention, perceptual attention, attentive memory, and persistent memory.

The MEMTRAX Memory Test has particular utility to detect memory dysfunction in subjects otherwise appearing normal that have very early Alzheimer's disease or other related types of dementia.

Method to Assess Retentive Memory—MEMTRAX Memory Test The MEMTRAX Memory Test is a method to assess retentive memory. The correct response of a subject which is examined is an indication that a particular image on a slide is a repeat of an image from a prior slide. The indication is most simply a press of a button, usually either the space bar on the computer keyboard or a touch of a touch sensitive screen as on a mobile phone. The indication may also be pressing one of two buttons, one to indicate recognition of the picture, one to indicate non-recognition of the picture. Other types of overt behavior may also be used as indicators of response, including any movement or vocalization.

Performance is tallied as correct recognitions (percent correct indicates retentive memory function) and correct rejections (percent non-responses or non-recognition indicators to initial presentations indicates level of perceptual attention to the stimuli and the validity of the memory assessment value). Signal detection theory provides measures d′ which is an estimate of discrimination between non-target and target items, beta which an estimate of the tendency to be more likely to respond to a target than a non-target, and C which is a measure of the tendency to respond as opposed to not respond (more independent of the d′ measure than beta). Receiver operator characteristic analysis uses sensitivity and specificity measures to determine how much information is provided by a test given a range of cut-offs for making a particular decision. Item Response Theory provides information about the performance of individual test items with respect to difficulty, discriminability and goodness-of-fit, with an established method to use this information to estimate a subject's level of function (as used by the Scholastic Aptitude Test and IQ tests). Neural Network Model analyzes each item for its weighted relationship to the outcome measure in the context of all of the other items.

Reaction time can also be measured. Measurement of the reaction time to a response to a repeated picture indicates how quickly the subject is able to process, perceive, and recognize the information as a repeated item. If measurement is made of reaction time to new pictures, the reaction time indicates the time which the individual processes and perceives information and makes an attentive decision that the information has not been perceived previously during the test.

Another way that the test can be administered is by showing slides to an audience. Audience members can respond to repeated slides by indicating that a numbered slide is a repeat of a previously shown image. The audience member can make this indication either by marking a numbered sheet (attached paper) or by pushing a button on a “polling transmitter”.

The test may contain any number of images, but works well with as few as 40 slides and 15 unique pictures. A second administration with a different set of stimuli in a different order can be used to confirm or further specify the levels of the various aspects of memory function. Alternatively, a longer test can be administered in which hundreds of slides are shown, with repeated slides interspersed. Between the initial presentation of an image and the repetition of the image, the test must contain a minimum of one intervening images, usually 5 to 20 images, and possibly over 200 images. Images may be repeated additional times, providing some items easier to recognize for more impaired and older individuals, so that performance in impaired ranges can be precisely assessed.

Key features of the MEMTRAX Memory Test design are:

1. A variable N-back design memory test for a series of stimulus presentations. For testing of retentive memory, N must be at least 2, possibly over 20, and is variable, with an average of at least 5.
2. This N-back design memory test can use photographs of objects and other images.
3. The specific photographs and electronic images dedicated to this test.
4. The specific images and photographs include, but are not limited to, representations of nameable and unnameable objects in the environment, scenes of any kind, line-drawings, abstract representations, text, and combinations of these. The specific images and photographs extend to faces (male and/or female), words (nouns, verbs, etc., abstract, concrete, simple, complex) and line drawings which vary from concrete, nameable to abstract and un-nameable.
5. The duration of a single test is from 30 seconds to 30 minutes. Optimal implementation is 3 minutes for elderly individuals. The test has a multitude of similar versions, so that the test can easily be repeated several times without the individual seeing repeated stimuli.
6. In the N-back design, images are shown for an initial presentation and then are subsequently shown later in the series to see if the subject recalls having seen the image before in the series. The subject may respond with a simple button (bar) press, for either recognition or non-recognition, and other buttons may be used to discriminate these response options.
7. The reaction times of the responses are measured.
8. Scoring of this test is percent correct=true positive/(true positives+false negatives) and percent new identification=true negatives/(true negative+false positive). Reaction time is a performance measure. Other scoring methods include use of Item Response Theory, Receiver-Operator Characteristic Theory, and Neural-Network Analysis, which can enhance the precision and stability of the TEST results, both for assessing memory in younger individuals through a continuum of such measures to the purpose of detecting early Alzheimer's disease. Another analytic method includes signal detection theory.
9. Many memory tests have been developed and have been widely used. However, this test is unique in its design for automatic administration and scoring as a brief test, using interspersed new and repeated images and a specified order of display.
10. The TEST can assess a broad range of memory function. Different versions of the TEST may use longer display times (3 to 10 seconds) and specially selected, predominantly easily discriminated images, for the purpose of detecting mild memory problems in elderly individuals that are in the early stages of developing Alzheimer's disease. Alternatively, images which are more difficult shown for shorter display times (i.e., under 3 seconds) may be used to measure high levels of memory function, particularly in younger individuals.
11. The different versions of the MEMTRAX Memory Test may be directly compared due to the pre-tested comparability of various sub-categories of item bundles, so that an unlimited number of test versions can be available, while any version is able to reliably estimate an individual's memory on the specified continuum of retentive memory function. The memory function of an individual may be evaluated precisely over time and evaluated for small but significant changes.
12. The TEST is surprisingly well-received by subjects, who nearly unanimously agree that the TEST is fun to take (potentially more than cross-word puzzles or sadokus). Accordingly, subjects are willing to take the TEST repeatedly over a period of time, which increases the likelihood that a subject will take the TEST enough times for a significant change to be detected if it occurs.
13. The images used in the MEMTRAX Memory Test are unique in their grouping and selection for testing a broad range of memory function precisely.
14. The order with which the images are displayed is unique for measuring retentive memory and establishing validity of the measurement.

The MEMTRAX Memory Test involves looking at a number of images and indicating which are duplicated. In the Memtrax Memory Test, correct responses are defined before the slide show begins.

The instructions are: “(NUMBER) images will be shown. Carefully look at each image. When you see an image for the first time, look at it carefully and try to remember it. If you see an image that you have seen before, respond to indicate that you recognize the image.”

The administration of the TEST by computer to an individual requires the following:

1) A slide or text-file which specifies the directions for performing the test which will be shown to the test taker (the subject taking the test).
2) A file with the identifiers of the specific images to be shown.
3) A file which contains the order in which images should be shown.
4) An algorithm for preliminary analysis of the test data.
5) The database that will store and the system to analyze the test data.

Memory function generally deteriorates with age, and memory impairments are a commonly unrecognized symptom of dementia. The objective was to characterize an audience-based memory test suitable for simultaneous screening of a large number of individuals.

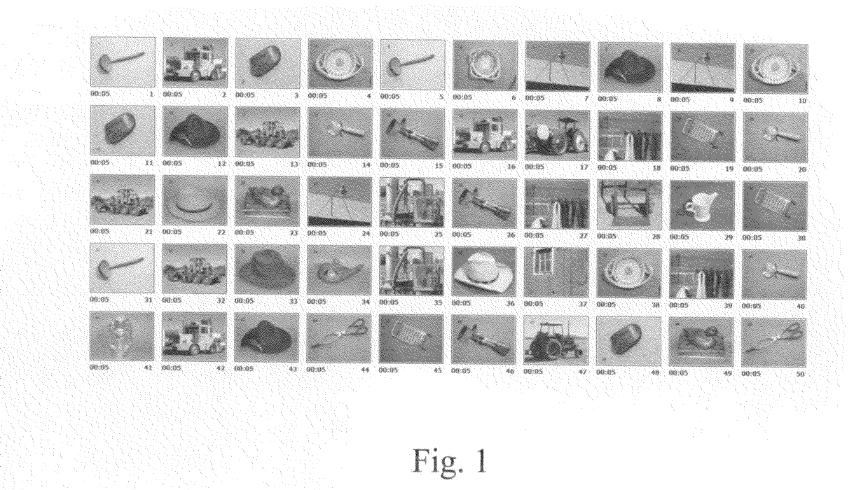

Referring to FIG. 1 the TEST can also be administered as a slide show (e.g., Microsoft PowerPoint), with the pictures and picture orders pre-established, each image shown for 5 seconds automatically, for which subject performances in an audience can be assessed either by a pencil and paper (e.g., audience members indicating the number of a slide which they think is a repeat) and by audience voting electronic technology, where audience members indicate if they think a picture is repeated, and the data for each individual's performance is stored in the administering computer's memory (e.g., Turning Point Technologies). The TEST was developed to assess recognition memory in audiences using a slide-show with 50 images, of which 25 are repeated. Audience members responded by recording if an image was a repetition. In test administrations to over 1050 participants, 868 individuals aged 40-97 years provided complete data.

Recognition memory performance as measured by discriminability (d′) showed a progressive, exponential decline with age and a progressive increase in variability. Individuals with low levels of education had lower scores than those with more education. Gender showed no effect. This audience-based memory test was sensitive to effects of both age and education. Such memory tests represent a practical approach to screen for early dementia and further development of this type of test is warranted. One critical issue is to define suitable cut-points for memory screen failure, since the same value is not appropriate for both young and old individuals. The cut-point should also reflect cost-worthiness of screening relative to age.

Memory impairment is often the most disabling feature of many pathological processes including neurodegenerative diseases, such as Alzheimer's disease (AD), stroke, etc. (Mesulam, 2000; Newman et al., 2001). Epidemiological studies indicate that 5% to 15% of adults aged 70 and older exhibit signs of dementia, and memory impairment is a core feature of dementia (Hy & Keller, 2000). Despite widespread understanding of the significance of memory disorders, they often go unrecognized (Ashford et al., 2007; Callahan, Hendrie, & Tierney, 1995; Finkel, 2003; Ross et al., 1997; Sternberg, Wolfson, & Baumgarten, 2000; Valcour, Masaki, Curb, & Blanchette, 2000).

Several factors interfere with the detection of memory impairment associated with dementia, including a failure to screen, avoidance of this difficult problem by affected individuals and their health care providers, and under use of available testing methods. With changes in the delivery of health care, physicians must work under strict time constraints, leading many physicians to not routinely screen their patients for dementia (Lawrence et al., 2003). One solution to the failure to detect memory impairments is to implement large-scale community memory screening. Numerous approaches have been advocated to screen for memory problems in the community (Ashford & Borson, 2008), and studies demonstrate that such programs can detect such individuals (Crews, Harrison, Keiser, & Kunze, 2009; Lawrence, Davidoff, Katt-Lloyd, Auerbach, & Hennen, 2001; Lawrence et al., 2003). However, community screening programs are logistically difficult. Currently available memory tests must be administered by a trained psychometrician in a one-to-one interaction in a confidential, quiet environment. Such tests are expensive to administer and uncomfortable for the individuals taking the tests, leading to poor motivation for repeat testing.

Audience-based methods for testing large numbers of individuals simultaneously have not been widely developed as cognitive screening tools. However, such tests could be used to screen groups of people for memory problems in order to identify high-risk individuals for further evaluation. Because memory impairment associated with dementia is commonly undiagnosed, a simple audience-based memory test designed to detect patients with early dementia would be valuable.

A significant issue is how to assess the utility of an audience-based memory-screening test to detect early dementia. Any population of older adults will contain individuals with diverse memory impairments. However, AD is the most common form of dementia, accounting for approximately two thirds of all dementia cases (Alzheimer's Association, 2010). The initial symptom of AD is typically a prominent amnesia in which the core symptom is difficulty in encoding new information (Ashford, Kolm, Colliver, Bekian, & Hsu, 1989; Salmon & Bondi, 1999). Even when patients with early AD can perceive and immediately reproduce new information (e.g., repeating a series of words), many neuropsychological studies have shown that the encoded information is easily lost under conditions of delay or interim distraction (Ashford & Schmitt, 2001; Elias et al., 2000). This specific type of memory impairment in AD has led to the suggestion that AD pathology is specifically affecting basic mechanisms subserving neuroplasticity (Ashford & Jarvik, 1985; Teter & Ashford, 2002).

The process of memory encoding can be tested in several different ways. However, recognition memory tests are especially suitable for this purpose as they provide the target stimuli within the test framework. Poor performance on a test of recognition memory provides strong evidence for an underlying encoding impairment, raising the possibility of an emerging Alzheimer process (Lowndes & Savage, 2007). In contrast to individuals with early AD, healthy adults can quickly and accurately encode massive amounts of new information. For example, landmark studies in the 1960s, 1970s, and 1980s demonstrated that healthy individuals perform well above chance on tests of recognition memory after viewing thousands of images for a few seconds each (Shepard, 1967; Standing, 1973) and after viewing highly complex images (Wright, Santiago, Sands, Kendrick, & Cook, 1985). Taken together with the encoding deficits found in early AD, these studies suggest that recognition memory tests should provide an effective screen for early AD. It is important to note that even though recognition memory has a huge capacity in healthy adults, it is nonetheless vulnerable to age-associated cognitive decline and increased age is associated with lower levels of performance (Grady et al., 1995; Schacter, Cooper, & Valdiserri, 1992). The aim of the current study was to measure AD-related memory performance in an audience population. For this purpose a variable N-back task (VNBT) was designed to detect memory problems in audience members. The VNBT was designed to be interesting in order to maintain audience attention.

The current study sought to characterize this repeat detection task and evaluate age-related changes in recognition memory in order to determine normal performance ranges. The VNBT performance was expected to decrease with age.

The VNBT was administered to over 1050 subjects between July, 2007 and June, 2008 at 26 sites (community events, senior citizen centers, retirement living communities, etc. in San Francisco Bay Area). The audiences ranged from 9 to 142 individuals (M=39; SD 34; range 9-142). There were 940 subjects who appropriately performed the memory test, and of these participants, 868 individuals provided three specific demographic items of information: age, education, gender (age: M=75.9 years old; SD 11.4; range 40.0-97.6; education: M=16.1 years; SD 2.52; range 6-21; gender: 68.7% female). In this group 86.6% of the participants were reported being “white”.

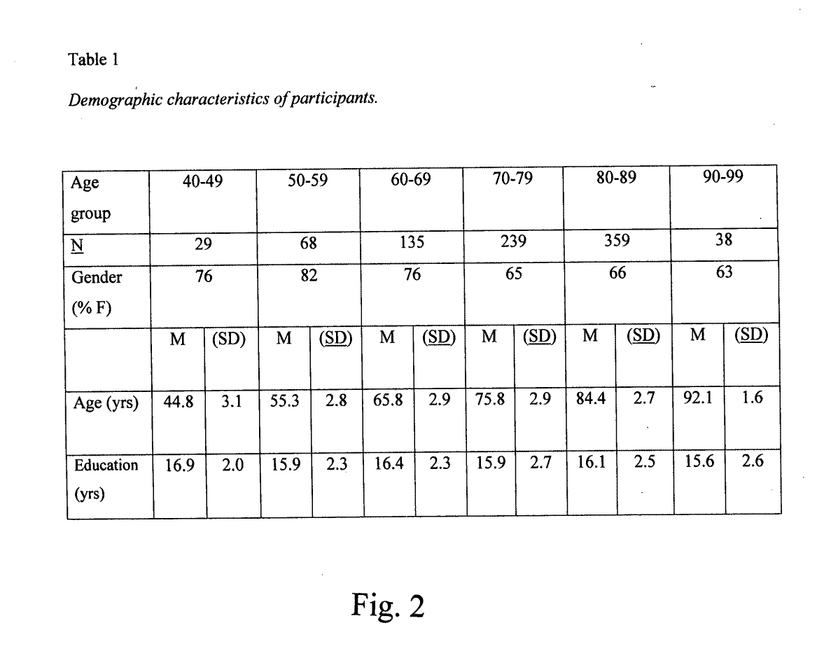

Referring to FIG. 2 participants were divided into six sub-groups according to age. Education level declined by 1.3 years from 16.9 to 15.6 from the youngest to the oldest age group, though the variation did not reach statistical significance (F 5, 867)=1.93, p>0.05). All age groups contained more females than males. The groups varied significantly in the number of males to females (N=868)=12.9, p=0.02).

The audience-based memory test was developed for testing recognition of easily remembered images. A “variable-N-back task” (VNBT or repeat detection after multiple intervening stimuli) format was used with numerous complex visual stimuli. Generally the images were of discrete objects, though similar objects and difficult to name objects were used to avoid strict reliance on verbal cues, provide a challenge, and maintain the interest of the subjects (the assortment of images was developed over several years). This approach reduced the ceiling and floor effects (only 8% of the subjects had a perfect score). Although audience testing is used widely in educational assessment, such testing procedures are unusual in cognitive neuroscience or clinical research. Accordingly, a primary aim of the study was to demonstrate that a recognition memory test can be administered to a large number of individuals simultaneously.

Twenty-five color images (digital camera) of manmade items were selected from a range of pictures. From these 25 items, a 50-item recognition memory test was constructed in the following way. The 25 items were first arranged in a random sequence, with repeated images interspersed. Fourteen of the items were one-time repeats and were inserted among the initial presentations of the test items.

Eleven of these items were shown for a third time, making recognition easier for subjects with impaired memories, providing more learning regarding a particular stimulus set, and allowing a comparison of first repeat recognition with second repeat recognition. The order was arranged such that there was an average inter-repetition-interval to the first repeat of 7.93 items (range=2 to 25 intervening items). The eleven items that were second repeats were inserted into the test with an average inter-repetition interval of 21.1, (range=10 to 36 items between the second and third presentations). The eleven un-repeated test items served only as foils.

The 50 items were numbered in sequence (1-50) with a large numeral in the top left hand corner and transferred to a PowerPoint presentation. A second series of ten items was constructed using similar color images and was used as a practice test before the full test was given (5 images, 3 repeated once, 2 repeated a second time). The need for such a practice test had become obvious during pilot work, which indicated that about 10% of audience members could not follow the verbal instructions on the first try. Participants were provided with a single sheet of paper. Demographic information was collected on one side of the page (age, education and race) and the other side was used as an answer sheet for the recognition memory testing. The answer sheet had columns of numbers corresponding to the 10 slides of the practice-test and the 50 slides of the full test. A single circle was adjacent to each number on which a subject could indicate their response by filling in the circle, and the sheet was organized so that it could be scanned for data entry.

Testing at all sites adhered to a standard format, which began with a 20-minute introductory talk, with slides about Alzheimer's disease and the signs of dementia. As part of the talk, all participants were offered the VNBT memory test, and the audience was told that participating in the memory test was optional, but that individual test scores would be provided anonymously at the end of the presentation. A statement outlining the subjects' rights was provided to all audience members on a written page and reviewed on a slide (Protocol approved by Stanford University Institutional Review Board; no identifying information was collected, and therefore written consent was not required). The same 10-item practice test and 50-item memory test were used at all sites (2 individuals publicly acknowledged taking the test before, but were not identified).

The VNBT was presented by projecting test items onto a screen using a laptop computer and projector. No effort was made to assess visual acuity of audience members or to assure adequate visibility from all parts of the room. However, the slides were generally easily seen from all vantage points of every room in which the test was administered.

Participants were told that they would see a series of 50 pictures one at a time for 5 s per image (no inter-image interval). They were instructed to look at each picture carefully and any time they thought an image was repeated they should note the image number shown in the top left hand corner and immediately mark the circle corresponding to that number on their answer sheet. No response was required if they thought an image was not repeated (i.e., novel). The 10-item practice test was given first. The presenter then addressed any questions relating to the test procedure, and then the full 50-slide test was given (250 seconds). After the test, the participants handed their papers to the rater to be scored. A rater scored each participant's answer sheet, after which the scores were returned anonymously to each participant. If scores indicated a high probability of memory problems, a notation was made on the anonymous score sheet encouraging the subject to visit their clinician for further evaluation (note that it has been reported that about 50% of individuals receiving positive screens will accept such a referral—Boustani et al., 2005). Results from the VNBT were analyzed using the correct and incorrect response information. The correct recognition rate (hit rate) and the false positive rate were used to determine a signal detection parameter, discriminability score (d′) (Green & Swets, 1966). The correct recognition scores included responses to only the first repetition of the items (n=14), while the false positive applied to all 25 items. A standard correction was necessary when calculating d′ values if the hit rate or the false positive rate were 100% or 0%. Following MacMillan and Creelman (MacMillan & Creelman, 1991), we converted 0% to 1/(2N) % and 100% to 1-1/(2N) % where N=the number of items. VNBT scores were analyzed in two ways. First, the relationship between individual test scores (d′) and age was examined using regression analysis. Next, three-way univariate analysis of variance (ANOVA) was used to examine the effect of age, education, and gender on errors and d′ scores. To determine the effects of age on test performance, participants were divided into six age-groups (see Table 1). To determine the effects of education on test performance, participants were divided into five groups corresponding with major divisions of attainment in the U.S. educational system [i.e., <12 years (high school), 13-15 years (some college), 16 years (college completion), 17-19 years (masters degree), and 20-21 years (advanced degree)]. Significant effects were investigated using the Tukey Studentized Range/HSD post-hoc test procedure to identify homogenous subgroups.

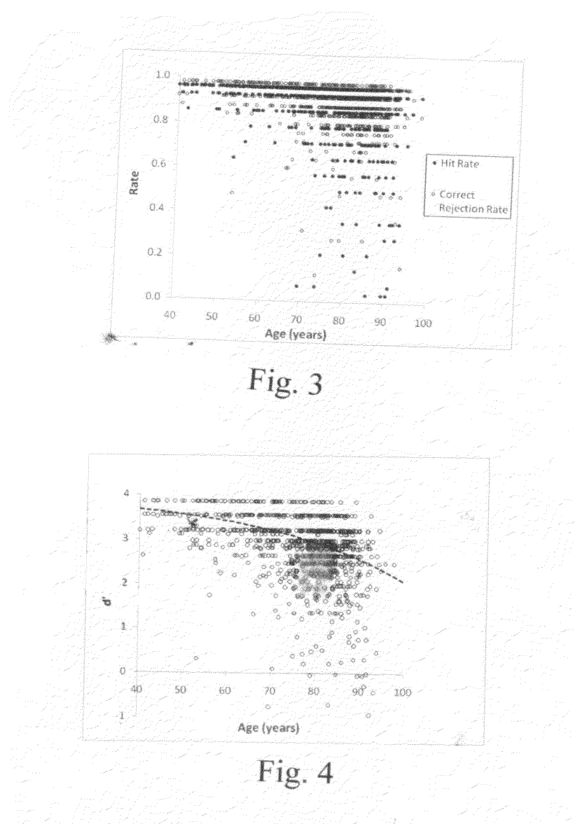

Referring to FIG. 3 increased age was associated with a significant increase in both miss rate and false alarm rate. The corresponding hit rates and correct rejection rates decreased with age as error rates accelerated in the oldest individuals with a non-linear relationship to age. Data were analyzed using nonlinear exponential and logarithmic data transforms. Error rates were found to be best explained using an exponential model (i.e., r2 of the regression was higher fitting an exponential transform than fitting a straight line). Regression analysis showed that miss rates increased significantly with age, exponential trend, F (1,866)=64.2, p<0.001; r2=0.069, beta=−0.02, constant=0.017, and likewise, false alarm rates increased significantly with age, exponential trend, F (1,866)=129.3, p<0.001; r2=0.130, beta=−0.026, constant=0.01.

Referring t FIG. 4 there is a relationship between the individuals' d′ values and their ages. Regression analysis revealed that test d′ scores decreased significantly with age, linear trend, F (1,867)=138.5, p<0.001; r2=0.138, beta=−0.026, constant=4.81. Data were also analyzed using an inversion of d′ scores (subtracted from the maximum value, 4), then regressed with an exponential model. This procedure explained the variance in the test scores better than a linear regression (r2 linear trend=0.138, r2 exponential trend=0.144), and regression analysis of the d′ inversion scores revealed that they decreased significantly with age with greater explanation of the variance than the linear model described above, exponential trend; F (1,867)=145.7, p<0.001; r2=0.144, beta=0.026, constant=−0.81. The 3-way ANOVA revealed significant main effects of age (F(5, 811)=12.97, p<0.001) and education (F(4, 811)=5.46, p<0.001) on VNBT scores. No significant effects were found for gender (F=0.62, p>0.05). No significant interactions were found between age, education, and gender (all Fs <1.2, all ps >0.05). For post-hoc test analysis, homogenous subsets were examined: six age groups by decade from 40 to 99 and the 5 education groups described above. Additional separate analyses for age were performed using participants having more than 12 years of education since there was no significant education effect noted above 12 years.

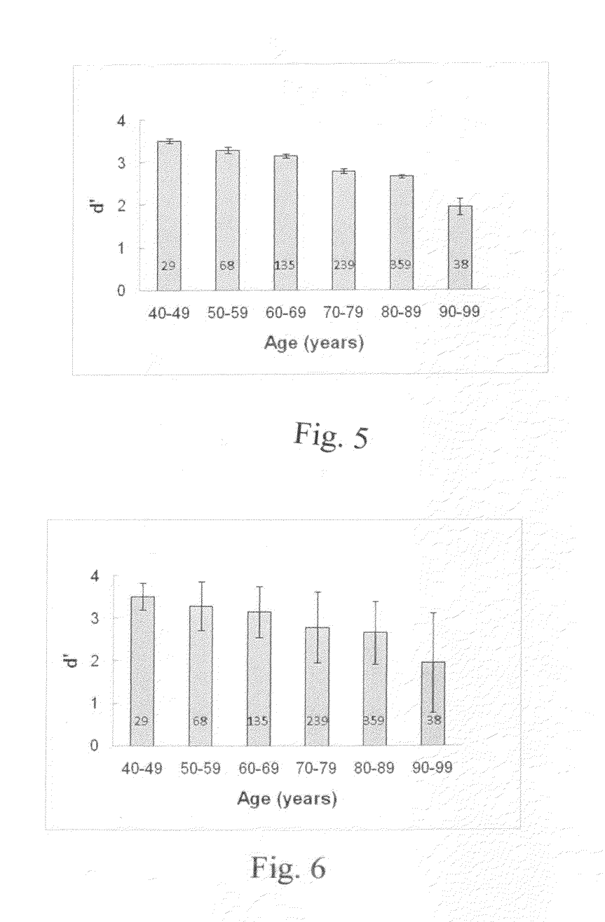

Referring to FIG. 5 in conjunction with FIG. 6 ANOVA results indicated that the VNBT was sensitive to age and was more difficult for older adults than younger adults. Again, age-associated effects were investigated further by examining the error rates: the missed items and the false positive rates that contributed to the overall d′ scores. Increased age was associated with significant increases in both the miss rate (F (5,867)=14.10, p<0.001) and the false alarm rate (F (5, 867)=13.96, p<0.001). Test discriminability gradually but significantly declined numerically with increasing age. Of note, the standard deviation of d′ performance increased progressively with increasing age.

Although the six age groups did not differ significantly in number of years of education (see Participant section), it was noted that the oldest group also had numerically the lowest average level of education.

In order to verify that the effect of age on the VNBT performance was not confounded by educational level, participants in the group with the lowest level of education (i.e., <12 yrs of education, n=82) were excluded in a secondary analysis. Results were essentially the same as when all participants were included. These data strongly indicate that test discriminability declined significantly with increasing age, F (5,785)=26.20, p<0.001. Again, increased age was associated with significant increases in both the miss rate, F (5,785)=11.48, p<0.001, and the false alarm rate, F (5, 785)=13.30, p<0.001.

The post-hoc subsets consistently showed exponential declines of performance with age. To confirm this effect, data were further analyzed with post-hoc Tukey tests, which automatically correct for multiple comparisons. This analysis supported a significant decline in discriminability with increasing age. Significant differences in test scores were found between the following groups of participants: 40-59 yrs, 50-69 yrs, and 70-89 yrs. The group older than 90-99 yrs of age was significantly worse than all other groups. Further, the miss rate was not statistically different within the groups with age range 40-79 yrs or 50-89 yrs, but showed a significant increase in the age group 90-99 yrs relative to the younger groups. False alarm rates showed a similar pattern and were homogenous within each of the following age ranges: 40-69 yrs, 60-89 yrs, and 90-99 yrs. When the individuals (n=82) with education less than or equal to 12 years were removed, the post-hoc tests showed that test performance expressed as d′ was similar across the age ranges 40-59 yrs, 50-69 yrs, 60-79 yrs, 70-89 yrs, and 90-99 yrs. The miss rates showed a similar pattern as for the previous analysis and were not statistically different within the age range 40-89 yrs, but the age range 90-99 yrs showed a significant decrease relative to the younger ages. False positive rates showed a similar pattern to the previous analyses and were homogenous within each of the following age ranges: 40-59 yrs, 50-69 yrs, 60-89 yrs, and 90-99 yrs.

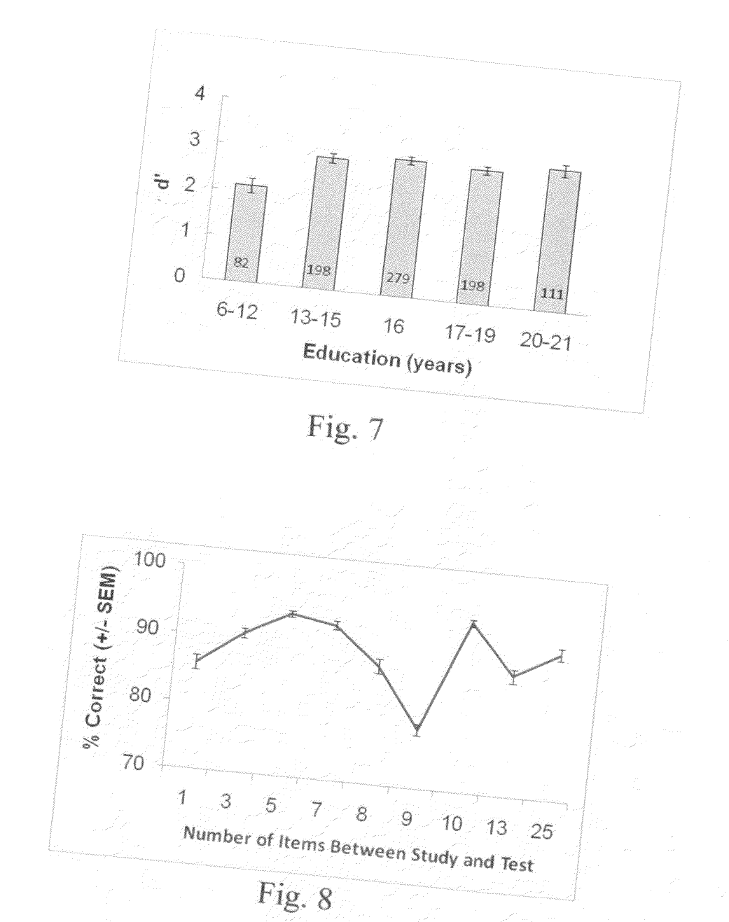

Referring to FIG. 7 there is an effect of education on test performance. Test performance was lower for those with education levels of 12 years or less relative to those with more education. However, performance reached a plateau after 12 years of education above which no significant improvement in performance was seen. Post-hoc tests showed that the test scores of the group with 12 yrs of education were significantly below all other groups (i.e., 13-21 yrs of education). A one-way ANOVA confirmed that the mean age did not vary significantly across the five education groups, F (4,867)=2.15, p>0.05. However, the lowest educational group (<12 yrs) was also numerically the oldest (79.5 yrs old vs. group mean of 76.4 yrs old for those with over 12 yrs of education), suggesting that levels of education vary systematically with age, and the poorer performance of the lower education group may actually be due to an age effect. In order to demonstrate that the effects of education on test score were associated with low levels of education (i.e., <12 yrs of education), the analysis was repeated using only individuals having more than 12 years of education. This ANOVA showed that when individuals with low education were excluded, no significant effects of education on test performance were found, F (3, 785)=1.65, p>0.05. Due to the repeat-detection format of the VNBT, participants were required to hold items in memory for a variable delay.

Referring to FIG. 8 the inter-repetition-interval ranged from 2 to 25 images. This delay could disrupt recognition performance in two ways. First, as the number of intervening items increased, the time delay between the first and subsequent presentations of the same item could reduce recognition. Second, as other test items were presented during the time delay, interference could build up across the delay. To explore these effects, a linear regression analysis was performed between the number of intervening items and percent correct. No significant relationship was found between the number of intervening items and recognition performance, F (1,8)=0.10, p>0.05, r2=0.02, beta=0.12, constant=88.6. The inter-repetition-interval had little overall effect on recognition and performance was maintained at a high level across repeated items (average=89%).

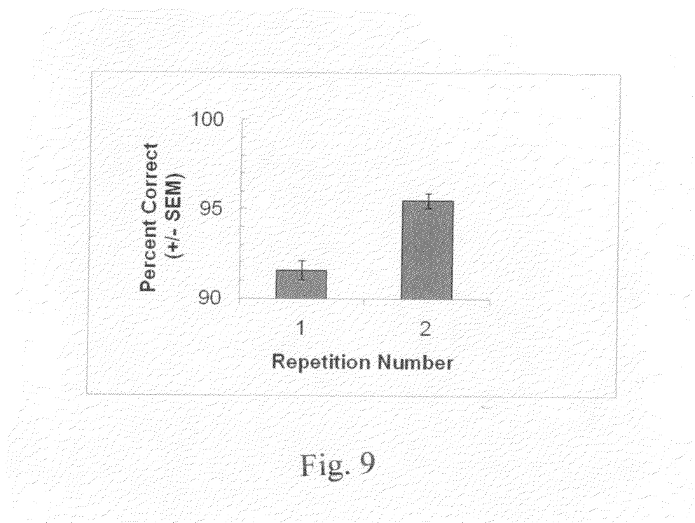

Referring to FIG. 9 another issue related to the repeat-detection format is that when test items are repeated multiple times, each subsequent presentation serves as a retrieval cue to reactivate and strengthen the memory representation of the information stored during earlier study (Thios & D'Agostino, 1976). In the current test, eleven items were shown three times, and recognition performance did increase across repeated presentations. A paired t-test compared the mean percent correct between the first and second repetitions and showed that this difference (91.6% vs. 95.5% correct) was statistically significant, t (867)=−10.30, p<0.005.

The results from this study of community audiences show that memory can be measured in a large group setting. The decreased memory with age found in this study is consistent with the general pattern of age-related memory loss (Crook, Larrabee, & Youngjohn, 1993; Salthouse, 2009; Schacter et al., 1992; Schaie, 2009). The present study focused on memory for complex information retained after a delay. In this test, memory storage was assessed using a recognition format. The experience presented here with this VNBT indicates that it is feasible to test audiences of older individuals for recognition of this type of information. This VNBT provides a strong assessment of the type of memory, frequently referred to as declarative memory (Squire, Stark, & Clark, 2004), which is impaired in Alzheimer's disease. Impairments in declarative memory are often found to be among the first symptoms during the progression from normal aging to AD (Ashford, 2008; Mickes et al., 2007). The observation that declarative memory is selectively impaired early in AD is consistent with the finding that the neurodegeneration associated with AD begins in the medial temporal lobes (Braak & Braak, 1996; Delacourte, 1999), an area of the brain known to be important for encoding declarative memory (Squire & Zola, 1996). Further, several studies have suggested that the specific aspect of memory most commonly impaired in preclinical AD is a difficulty in encoding new information (Ashford et al., 1989 and 1995, and Ashford, 2008; Salmon & Bondi, 1999).

Accordingly, this VNBT is well suited for measuring memory most relevant for the detection of the early memory difficulties found in AD patients.

Memory encoding can be tested in many different ways. Recognition memory is particularly suitable for the assessment of encoding as the target stimuli are given as part of the test materials (Lowndes & Savage, 2007). Thus, recognition memory is less dependent on retrieval processes than are other commonly used testing formats such as free- and cued-recall. Impairment in recognition memory is evidence for impairment in encoding which raises the possibility of an emerging Alzheimer's process (Wisdom, Callahan, & Hawkins, 2011).

Referring again to FIG. 5 and FIG. 6 from the testing in the population examined by this study, the VNBT appears to measure learning across a broad range of memory abilities. The analysis of the standard error of the mean (SEM) and the multiple statistical analyses of education showed the sensitivity of the VNBT to age-related changes in memory. The VNBT test also showed a substantial increase in the standard deviation of each sample group (SD) with increasing age, and therefore, the VNBT provides limits for estimating the statistical variation that would be expected at various ages. After establishing the expected variation in memory function for a specific age, abnormal memory function levels can be defined for individuals of a specific age. In showing the capacity to assess memory deficits, the VNBT shows potential for detecting memory problems that are indicative of early signs dementia related to AD or other disorders and could be used to screen populations for dementia.

There are specific issues that must be addressed when considering a test for screening. Performance which are poorer than 2 SDs below the mean for any population may be defined as abnormal. Younger individuals (e.g., 40-50 years) with performance levels which are poorer than 2 SDs below the mean for this age group should definitely be considered to be of clinical concern. The problem is that low scores in older individuals may lie within 2 SDs of the mean for their own older age group, and thus would not be “abnormal”. Accordingly, a further consideration is below what absolute memory performance level might individuals of any age be at risk for having functional impairment? These are two different approaches to determining cut-off levels that might be considered when using a test for screening purposes. However, when developing a screen for memory problems, it is necessary to consider cost-effectiveness (including consideration of the pathological entity targeted for screening). The decision about whether to screen an individual and the critical level for clinical concern depend on an analysis of many factors. The factors to consider for such an analysis include: incidence of disease; the benefit of a true-positive screen; the cost of a false-positive screen; the incidence of the target problems in the population; and the cost of the test (Ashford, 2008). A large factor in the decline of memory performance with age is likely to be the exponential increase of dementia incidence (Jorm & Jolley, 1998). The value of using a particular level of test performance as a positive screen for an individual is approximated by a cost-worthiness analysis (Ashford, 2008). This approach is more difficult than a simple cut-off value for screening as described above, but is better for addressing clinical needs.

The VNBT may be useful for detecting memory problems related to a variety of disorders. It is unclear whether the alterations in memory associated with early AD are different from those associated with age-related changes in memory. Further, AD itself may be a complex interaction of at least two pathological processes (amyloidopathy and tauopathy), which have different time-courses (Morris et al., 2010) and roles in different aspects of memory impairment that are otherwise considered “normal aging”. Further, for proper screening of older individuals for cognitive impairment, more issues than just memory performance need to be considered.

Performance levels on a test like the VNBT could be monitored over time to detect indicative of a progressive cognitive disorder. An important issue for this VNBT is that the test is “fun” so that individuals may be willing to take the test repeatedly. Changes over time should be assessed with respect to age-cohorts since normal performance levels and changes over time do vary according to age (Schaie, 2009). Of note, the pattern of memory deterioration with age was best fit by an exponential model, suggesting that the underlying aging physiology follows the Gompertz Law, which states that the rate of system failures increases exponentially with age, in this case, the failure of mechanisms subserving the performance of this memory task (Ashford et al., 2005).

The VNBT is not affected by education level beyond high-school, probably because the memory processes targeted by this test require relatively simple object-information storage and processing, similar to what laboratory animals can be trained to do (Ashford, Coburn, & Fuster, 1998; Mishkin & Appenzeller, 1987; Wright et al., 1985). Accordingly, education appears to have at most a minimal effect to confound assessment. Subjects with a high-school education or less performed significantly less well, and there needs to be further study of individuals with low education on this test.

Another issue is how effective is the VNBT for assessing information encoding? It is well established that there are significant declines in delayed-recall performance as individuals get older (Petersen, Smith, Kokmen, Ivnik, & Tangalos, 1992). Much accumulated data indicate that these differences pertain to the fact that it takes older individuals longer to learn new information (encoding), but once learned, it is retained well over numerous delay intervals e.g., (Craik, 1971; Wickelgren, 1975). For example, if one compares the decline on recall scores from immediate to delayed recall, there are no statistically significant age-related differences (Petersen et al., 1992). Thus, if one allows healthy older subjects to learn material well (i.e., to the point where few errors are made), they do not forget what they have learned more rapidly than the young. However, if healthy older subjects are not given the ability to learn material to the same level of proficiency as younger individuals, after a delay, less information on average will be retained by the older person (Albert, 1996). Based on these prior findings, there is a question about whether older individuals may have performed better if the stimuli were presented for a longer period of time (i.e., more than 5 seconds per image).

Widespread individualized testing of older subjects to screen for memory difficulties has not been practiced in the past. However, it is important to screen older people for memory problems that may indicate dementia (Ashford et al., 2007). While screening tests are widely used throughout Medicine, they are not yet recommended for dementia or Alzheimer's disease (Boustani et al., 2005), and this lack of recommendation is based in large part on the lack of an easily administered and validated screening tool. The VNBT test presented here could be developed to serve this important need.

Clinically, any screening test must be seen as a preliminary assessment and definitely not a diagnostic test. However, the use of a test to screen for memory problems is appropriate (Ashford & Borson, 2008). Further, the VNBT approach offers a process that can be adapted for many settings and cultural milieus.

In summary, a brief, easy to administer test for audience or large-population administration was found to have significant sensitivity to the changes in memory accompanying normal aging and could form the basis of a screening system to detect memory problems indicative of clinically significant memory disorders. Since the VNBT measures the type of memory most affected in early AD, this test could serve as a practical approach to screen for early dementia associated with AD. Further development and study of this type of testing and population assessment is warranted.

This specification also provides for the presentation of visual stimuli (pictures or words) using a computer:

A specific test is defined by its instructions which appear on a specific image file (the “instruction sheet”).

The stimuli are specifically referenced by a file (the “images index file”, which is stored in a specific directory, e.g., Isets) which indicates the address locations of the stimuli.

The order of the stimuli is specified by a second file (the “order file”, which is stored in a specific directory, e.g., Osets).

The stimuli are presented in the order specified by the “order file”.

The duration of stimulus presentation is set for a specific test administration.

(OPTION) the stimulus presentation duration for an individual subject can be set for a specific individual and can be set to vary during the administration of the test according to the performance of the subject.

Responses may consist of a single indication from the subject taking the test and may include the press of a key (e.g., the “space bar”), any other similar response or measurable movement, or an utterance (detected by a microphone).

(OPTION) secondary responses may be instructed (e.g., for indicating that the subject decides that the displayed image is new), using a different indicator (e.g., left-arrow for old versus right-arrow for new), and reaction time to the second indicator may be measured as well.

The computer detects the responses (or lack of responses to a specific stimulus within a certain period of time) and records the reaction time with millisecond precision for each stimulus presentation.

The reaction time (response data) from each individual stimulus presentation is recorded for subsequent analysis. If a reaction is indicated within the observation window, the precise reaction time is recorded. A response is a reaction time within the observation window and is correct or not according to the instructions specified for the test.

Analysis of the data for presentation of summary results can occur immediately following the response (or lack of response) to the last stimulus or may be done at any later time.

The platform administering the TEST (computer program, slide-show) may also be used to show tests for:

Simple reaction time (the image set is a single content image and blank image, or a series of images, with the order set indicating that correct response is responding to the content image—or blank image, depending on the cognitive function being assessed).

Choice reaction time (the picture set contains two or more content images, and the order set indicates that a response is required only for a specific image or defined set of images. Alternatively, if two types of response can be made, alternate responses may be instructed as correct for each of the images or defined sets of images.

The “Super-Simple” Reaction-Time Test (a test developed by Dr. Ashford in 1986 that has the response instruction included in the stimulus itself, e.g., an arrow indicating which way to respond.)

N-back Attentive Memory (1-back, 2-back, or 3-back: in this well-established testing paradigm, the correct response is to the repetition of an image shown 1, 2 or 3 images before the most recent image. This paradigm is a test of Attentive Memory”.

Continuous Performance Task (In this well-established paradigm, a series of images is shown with a rare target image occurring which requires a response. This paradigm is a test of General Attention).

These ancillary tests may be used for determining impairment of a variety of cognitive functions. In many patients with mild cognitive impairment or mild dementia, only retentive memory is impaired and other tests of cognition are preserved.

A computerized test may include a tapping speed test, which can also help to determine if the subject's movement functions are in the normal range. This information can be used in adjusting the interpretation of the reaction-times measured in the test, to distinguish the component of the speed related to movement function from the component related to cognitive function.

Images may be complex pictures that can be easily named or not named. Images may also be words that are easily visualized as nameable objects or not easily visualized (abstract, emotional, complex).

The working model for the Memtrax Memory Test uses picture images in which there are 25 new pictures and 25 repeated pictures. For each set of pictures, there are 25 total pictures divided into 5 bundles of 5 pictures. All pictures are from real, color photographs, no black & white, no line drawings, no sketches.

The 5 bundles are selected to follow these categories:

A) Abstract, difficult to name, still-life, landscape, rocks, minerals, etc.
B) Buildings: houses, shelters, etc.
C) Clothing: hats, shirts, apparel, etc
D) Female oriented: kitchen ware, sewing items and furniture.
E) Male oriented: trucks and tools.

For these bundles, there are 5 categories that have been developed:

A) Nature: landscape, rocks, minerals, water, flowers, difficult to name.
B) Buildings: houses, barns, fences, walls, windows, outdoor items.
C) Clothing: hats, shirts, belts, apparel, functional jewelry.
D) Kitchen, household: utensils, cups, bowls, furniture, sewing items.
E) Machinery: vehicles (trucks, boats), tools, equipment.

There are sub-categories under each category, then groups of 5 pictures under each sub-category. Among each group of 5 pictures, there are 2 items that are slightly similar, while the rest are easily perceived to be different.

There are no people, no animals, and no writing/lettering, with a general avoidance of inanimate animals and statues. There are no emotion-generating pictures, such as food, expensive jewelry, weapons, sexual material, burning, gruesomeness and gore. There are no pictures that would immediately be recognized by more than 10% of the population (Golden Gate Bridge, Taj Mahal, pyramids, etc.)

(note that these items could be used in alternative tests, but not a test focusing on objective, non-emotional memory.

Photographs of paintings or complex pictures are generally avoided, though they could be under the abstract bundle.

Unique cultural items are generally to be avoided, but can be selected for specific populations, following the same rules (e.g., Chinese dishware and furniture has been used for a presentation to be given in China).

These exclusions only apply to a version of the test for preliminary screening of older individuals for memory dysfunction. The excluded categories may be used for evaluating memory in younger individuals or specific areas of deficits in individuals of any age.

Order definition is the number of single, double, or higher repetitions, can be specified to modify test difficulty.

The pictures vary in color, and background color should have some variation. However, the pictures are clearly distinguishable independent of color. The pictures should be clear. They may be 20-80 K JPEG images—medium resolution, 320×240, with good quality and no noticeable pixilation. A high resolution version of the images of 640×480 or higher is permissible if the computer capacity is feasible for rapid down-load of the images and perceptually instantaneous presentation of each image. There can be several bundles that are similar, but not so similar that they can be confused from day to day across 20 image sets.

Pictures should be named by convention, A-E; category, sub-group, number, Example of name: A-WaterFall-01.jpg

There are five categories of bundled items and examples of groups of five are as follows:

A) Nature: landscape, rocks, minerals, water, flowers, difficult to name.
B) Buildings: houses, barns, fences, walls, windows, outdoor items.
C) Clothing: hats, shirts, belts, apparel, functional jewelry.
D) Kitchen, household: utensils, cups, bowls, furniture, sewing items.
E) Machinery: vehicles (trucks, boats), tools, equipment.

Among these 5 bundles, more specific descriptions are:

A) Nature: landscape, rocks, minerals, water, flowers, difficult to name are interesting rocks and minerals, landscapes including vistas and forests, waterscapes including lakes, ocean, rivers and waterfalls, flowers and flower arrangements.

Among buildings are houses, barns, fences, wall, windows and outdoor items, outdoor ornaments.

Among clothing are hats, shirts, belts, apparel and functional jewelry.

Among apparel are shirts, socks, shoes, slacks and blouse.

Among accessories are hats, belts, scarf, mittens, gloves and canes.

Among kitchen and household items are utensils, cups, bowls, furniture and sewing items.

Among kitchen wares are glass bottle, pots/pans, mugs, bags, champagne/wine glasses, crystal wares, sewing items, buttons, Jewelry (not stunning), hair items, Furniture: Desks, Chairs, Tables: side, end, coffee, dining, Chandeliers, lamps, Door knobs, Machinery: vehicles (trucks, boats), tools, equipment, Automobile/parts: Tire treads, Trucks, tractors, Ships, boats, Tools, Electronic Equipment: Speakers, Bells, and keys

The following is an example of an order of the 50 images include 25 new pictures (NEW) and 25 old pictures (OLD) that has been implemented in several computer platforms and an audience presentation platform. Rules are adapted from Gellerman, L. W. Chance orders of alternating stimuli in visual discrimination experiments. Journal Genetic Psychology, Volume 42, pages 206-208 (1933).

No more than four images of a specific type, NEW or OLD, occur together. No more than four alterations in a row (NEW, OLD, NEW, OLD). First 2 items are NEW. Last 2 items are OLD. In first 10 items there are 7 NEW items and 3 OLD items. In last 10 items there are 3 NEW items and 7 OLD items. In the middle three groups of 10, there are 5 NEW items and 5 OLD items in each group.

Twenty images are repeated once, 5 images are second repeats, and 5 images are not repeated (2 in last 10, and 1 in each of the middle 10).

Examples of orders of the items from the 5 bundles (all from a single sub-group within each bundle) within the 50 image presentations. One of each of the 5 bundles must appear as a NEW in the first 10 images. One of each of the 5 bundles must appear as an OLD in the last 10 images. For each of the 5 bundles, there must be one item for which the OLD item occurs at least 20 items after the NEW image. Each bundle has one item which is repeated after just one intervening stimulus. There are no adjacent images from the same bundle. Each bundle has one item shown 3 times (NEW, OLD, OLD), with the second old stimulus occurring after at least 10 intervening items. Each bundle has one item which is not repeated.

The Algorithm for Computer Program includes:

1) Show loading-progress indicator if more than 5 seconds for loading is possible.
2) Establish Fixed Variables (may be varied):
a. Length of permissible reaction time, shortest, longest (example for complex images: 150 to 2900 msecs)
b. Time for showing new images (start with 3 seconds)
3) Load Instruction Set, Image Set (sub-directory “Isets”), Order Set (sub-directory “Osets”).
4) Load Images (according to locations identified in Image Set).
5) Pre-load Images for display.
6) Begin client interactions—ask for indication of agreement with license.
7) Show instruction that ESC key may be pressed to end test at any time.

### 8) Enter “FULL SCREEN MODE”

9) Show Instructions, request SPACE-BAR-Press to begin test.

### 10) Enter Display-Response Routine.

a. Select image for next display.
b. Note computer time in milliseconds=start time.
c. Display image.
d. Display time=computer time−start time.
e. Monitor key-board for key press (or touch screen for touch)
i. If ESC key pressed—stop; query about restart with different test.
ii. If non-SPACE-BAR key pressed, wait for full allowed time to exit.
iii. If acceptable key board press, response time=display time.
1. if too short, wait for full allowed time to exit wait routine
2. If permissible time (e.g., 3 seconds), exit wait routine
f. Monitor display time
i. If display time less than allowed time, go to “d”, Display time.
ii. If display time greater than or equal to allowed time, exit wait routine
11) Exit display-response routine.
12) Check performance of subject (note option to change image display time)
13) Check image number
a. If image number=50, continue
b. If image number <50, go to #10, Display-Response Routine
14) Calculate performance of subject based on Order Set and Response Times
15) (OPTION) display data to subject.
16) Stored Data (may send to server)
Offer choice to end program or continue with new process. Computer Programs in which the TEST has been Implemented: Prototypes of the TEST have been written in:

HTML-Javascript

JAVA

FLASH

PHP

The test has been given in several versions as a PowerPoint presentation, to over 2000 individuals.

Screening for memory problems, particularly those associated with dementia and Alzheimer's disease, has presented a significant logistical problem. The currently available memory tests are time-consuming and generally must be administered by a psycho-metrician in a one-to-one interaction with a participant in a confidential and quiet environment. Such tests must trade duration and participant burden with poor accuracy and a low ceiling effect making assessment of normal individuals problematic. There is a need for a simple, accurate memory test that can be administered in a group setting that is feasible for testing older individuals.

The MEMTRAX Memory Game was adapted to a slide show format and an approach reminiscent of college aptitude testing for a large group. Over the course of two years, this format was used over forty times at various community events, senior citizen centers, and retirement living communities, with over 1500 participants tested. Between Jul. 1, 2007 and Jun. 30, 2008, the test was administered at 26 sites using a single sheet, demographic information on one side and on the other, an answer sheet on which participants could indicate recognition of repeated pictures, in a format that could be scanned for data entry and analysis. The answer sheet had pre-assigned identification numbers and columns with numbers and single adjacent circles. Participants were shown a series of numbered slides, 5 seconds for each. Participants were asked to fill in the circle next to the number on a repeated slide. After a brief introduction and a short practice test of 10 slides, the participants completed a 50-slide test, that had 25 unique pictures, 15 repeated once, and 10 of those repeated a second time. After the test, the participants handed their papers to the rater to be scored. While the rater scored each participant's answer sheet, a presenter answered audience questions, after which the scores were returned anonymously to each participant. (Protocol approved by Stanford University Institutional Review Board.)

Data were obtained on 1063 participants at the 26 sites (average 41 participants per site, range 8 to 142), mean age (for 697 participants) was 74.5+14.5 years, range 20 to 95, with 41 participants over 90 years of age and 52 participants less than 50 years of age. For individual participants, test results were scored as the overall percent correct, the number of false-positive errors, and the number of false-negative errors. Of 708 scored tests, 540 participants (76%) scored 90% correct or better, with 48 participants (7%) having perfect scores and only 8% scoring below 80% correct. There were 67 participants (10%) who had more than 5 false-positive errors (incorrectly indicating an image was a repeat), while the same number of participants, 67, had more than 5 false-negative errors (failure to recognize a repeated picture). Performance on individual images was also analyzed. Of the new images, 2 were missed 64% and 58% of the time (false-positives), with 7 being missed between 5% and 27% of the time (all in previously shown categories), and the remaining 16 of the new images were missed less than 5%. Of the repeated images, 2 were missed 33% and 20% of the time (complex images) and all the rest were missed 16% or less. Only 3 repeated images were missed less than 5%. Thus, the repeated image errors showed less variability than the variability of the errors on new pictures. These results suggest that particular items triggered false recognitions, but recognition failures occurred more uniformly across pictures. The effects of age were also analyzed. Percent true negatives decreased from 95% at age 50 to 85% at 95 years of age. Percent true positives decreased from 100% at age 50 to 80% at age 90 years. There were statistically significant associations of performance with age.

While the accuracy, reliability, and validity of this testing format has not been conclusively determined, generally, participants getting more than five false-negative responses are of concern for the presence of Alzheimer's type dementia and those getting more than five false-positive responses are suspected of having problems with attention or disinhibition suggestive of fronto-temporal dementia. The MEMTRAX slide-test is not reliable for those participants with visual impairment or problems limiting their ability to fill in a circle with a writing implement. However, the experience with this format is that it is well accepted by audiences and has the potential to provide highly accurate and cost-effective screening for memory problems.

In an era of increasing pressure to detect and manage prevalent disorders as early in their course as possible, screening has become an accepted norm for many conditions. If medical professionals and the public accept screening for hypertension, diabetes, breast cancer, and colon cancer, why is there no widespread demand to screen for dementia? Detection of dementia—the most disabling common condition of later life (Aguerro-Torres H et al., 2001)—is currently left to chance (Ashford et al., 2006; 2007).

Numerous approaches have been advocated to screen for memory problems, dementia, and Alzheimer's disease (Ashford, 2008).

However, most of the approaches involve direct testing of potential cases or questioning of reliable sources (case-finding). Many of the tests have poor sensitivity and specificity for dementia, are cumbersome to administer, and are generally unpleasant for the patients. There is a clear need for a screening system that is attractive to prospective users, both patients and clinicians, which can provide reliable information, including baseline evaluation and frequent repetitions. By focusing on memory function, a screening test can address the issue most important for recognizing the earliest indications of Alzheimer's disease, new-learning memory difficulties. Visual information provides an essentially unlimited challenge to the brain's memory storage mechanisms. Performance information can be used to determine when further testing is appropriate. The purpose of this presentation is to report on the experience with a computerized memory test system that was adapted to a Power-Point slide presentation to be administered to a group of subjects. Results are presented from administrations between Jul. 11, 2007-Aug. 14, 2008.

The principle psycho-pathological factor in Alzheimer's disease is the attack of the formation of new memory traces that can be retrieved after distraction (neuroplasticity, Ashford & Jarvik, 1985; Teter & Ashford, 2002). For example, recall of learned words after an interval is the earliest problem seen in Alzheimer patients (Ashford et al., 1989; Ashford & Schmitt, 2001). This process is commonly tested using several different memory challenges. However, providing complex stimuli that are easy for a normal person to remember would provide the most effective test for the Alzheimer process.

MemTrax was developed based on the concept of providing a large volume of easily remembered information to a subject, then testing the recollection. The format used is referred to a “long-N-back” paradigm, with multiple complex visual stimuli, based on work by Shepard, 1961. Generally the images are of discrete objects, though similar objects and difficult to name objects were used to avoid strict reliance on verbal cues and to provide a challenge and maintain the interest of the subjects.

The initial paradigm used a computerized administration format and then a web-based format. However, due to the difficulty in getting older individuals to participate in web-based games, particularly those individuals with mild cognitive problems, the MemTrax game was reformatted to a PowerPoint slide show, running automatically with 5 seconds presentations for each stimulus. 25 discrete objects are shown, with 20 of them repeated, 5 repeated a second time, making a total of 50 objects, requiring 250 seconds to display. The audience is given a formatted answer sheet and instructed to fill in the circles next to the numbers on the images which are repetitions.

The MemTrax test has been under progressive development since 2000. The current version was given between Jul. 11, 2007-Aug. 14, 2008 on 26 occasions to senior citizen groups and health-fair participants, with a total of 1018 subjects filling out the questionnaire and submitting it for scoring (at most venues, a few subjects watched without taking the test or did not hand in their answer sheet, but no count was made of these individuals). There were an average of 39 subjects completing the form at each site (range 9-142, stdev=34).

Data were entered with a scanner into a spread sheet format (REMARK software and EXCEL spreadsheet, results triple checked by hand). Analyses were computed from the EXCEL spreadsheet, which was also used to produce the graphs.

Data entered as of Dec. 2, 2008-1018 individuals from 26 sites collected and considered that the individual had been able to perform the test. 805 reported being “white”. 31<40y/o

Of the 1018 individuals that were considered to have taken the test in a fashion that could be scored (about 20 were eliminated, 31 were below chance (12/25 or less) on the true negative or true positive score (True—:3 males, 10 females True+:8 males, 11 females) (not included in graphs. Of these 1018 individuals, those scoring less than 80% correct for True−, 19 males, 51 females; for True+, 25 males, 54 females.

Of these 1018 individuals, those scoring better than 80% for True−, 276 male (93.6%), 602 female (92.3%); for True+, 270 male (91.5%), 598 female (91.7%).

Only 82 subjects had perfect scores, 230 made 1 error, 700 made 5 or fewer errors (about 70%), and 132 made 6-10 errors.

Plots are shown for the 858 individuals with age, gender, ed data, red is first presentation, green is repeat, males in blue, females in pink.

Performance on new images (True−) was more variable than performance on old images (True+). There is minimal difference in performance of individual items between males and females, in spite of significant “male-role” and “female-role” items. There is a significant decline of function with age, with the age-effect best explained by an exponential increase of errors with age (“Failure Theory”). Females had a greater association of false-positive errors with age than males, while the false-negative error association with age was similar by gender. Education was not significant in performance.

MemTrax is a brief, convenient, fun test of the type of complex memory affected by Alzheimer pathology. Recognition failure (False−) indicates failure of learning circuits—typical of Alzheimer's disease. False-recognition (False+) responses are indicative that the subject is not paying attention and is failing to inhibit the recognition response, thus more suggestive of other types of psychopathology, including fronto-temporal dementia. MemTrax can test many levels of memory impairment accurately, validly, and reliability. Alzheimer's disease is not a dichotomous diagnosis but a continuum of impairment best assessed probabilistically using Item Response Theory (Modern Test Theory)—(Ashford & Schmitt, 2001).

From the foregoing it can be seen that a method of testing memory for Alzheimer's disease has been described. It should be noted that the sketches are not drawn to scale and that distances of and between the figures are not to be considered significant.

Accordingly it is intended that the foregoing disclosure and showing made in the drawing shall be considered only as an illustration of the principle of the present invention.

What is claimed is:

1. A method for assessing memory in a subject, comprising the steps of:
(a) presenting to the subject a list of items to be retrieved from memory by the subject wherein the items presented to the subject are intermixed with repetitions of said items being tested for recognition;
(b) having the subject recognize the presented items from memory wherein the subject is tested to determine if he recognizes each repeated item as being a repeated item;
(c) determining the subject's response speed to each of said recognized repeated items wherein said response speed for each of said recognized repeated item is the time required between when the subject is shown a repeated item and when the subject responds that he recognizes said repeated item; and
(d) analyzing a plurality of said response speeds for said recognized repeated items.
